(19) United States
(12) Patent Application Publication
(10) Pub. No.: US 2024/0096200 A1
(43) Pub. Date: Mar. 21, 2024

(54) LIFE EARLY WARNING SYSTEM BASED ON SENSOR ACQUISITION

(71) Applicant: Beijing Huiyang Science & Technology Co., Ltd, Beijing (CN)

(72) Inventors: Zhenyu SHI, Beijing (CN); Xiuyan LI, Beijing (CN)

(73) Assignee: Beijing Huiyang Science & Technology Co., Ltd, Beijing (CN)

(21) Appl. No.: 18/234,555

(22) Filed: Aug. 16, 2023

(30) Foreign Application Priority Data
Sep. 16, 2022 (CN) CN202211128136.1

(51) Int. Cl. G08B 25/01; G06T 7/73; G06V 10/774; G06V 10/80; G08B 21/02; G08B 29/18

(52) U.S. Cl. CPC G08B 25/016; G06T 7/73; G06V 10/774; G06V 10/806; G08B 21/02; G08B 29/18; G06T 2207/20081; G06V 2201/07

(57) ABSTRACT

The present disclosure pertains to the field of health monitoring technologies, and discloses a life early warning system based on sensor acquisition, including a sign sensor, a camera, a location analysis module, an attachment location correction module, a sign detection module, an alarm feedback module, a detection optimizer, a medical comparison library, a medical platform, and a client, where the sign sensor is configured to attach to a specified location of a user body, and acquire user sign information in real time. In the present disclosure, a location of the sign sensor is analyzed by using a cascade network, which can ensure accuracy of an attachment location of each sensor, avoid reduction in precision of subsequent detection due to a sensor location abnormality, greatly improve detection accuracy, further prevent an abnormal sign report due to misusing by a child, and improve user experience.

### CROSS REFERENCE TO RELATED APPLICATION

This patent application claims the benefit and priority of Chinese Patent Application No. 202211128136.1, filed with the China National Intellectual Property Administration on Sep. 16, 2022, the disclosure of which is incorporated by reference herein in its entirety as part of the present application.

### TECHNICAL FIELD

The present disclosure relates to the field of health monitoring technologies, and in particular, to a life early warning system based on sensor acquisition.

### BACKGROUND

With the improvement of economic level and the gradual development of science and technology, the state gradually increases investment in residents' security, for example, establishes a medical insurance system for urban residents. In addition, people's health and medical awareness are gradually developing and improving. In this case, limitations of conventional medical devices that are not easy to carry gradually fail to meet requirements of people for home vital sign monitoring. With the rapid development of computer technologies and microelectronics technologies, and gradually standardized and normalized vital sign indexes, a home life early warning system becomes a reality. People can continuously and dynamically monitor and early warn human vital signs in daily life and a clinical environment, greatly reducing interference of sign detection on normal human activities.

After searching, Chinese Patent No. CN111643069A discloses a health early warning wearable device based on vital sign monitoring and analysis. The present disclosure overcomes inconvenience caused by great time consuming and poor mobility in working of vital sign signal acquisition; implements real-time storage, display and analysis of data on a server and a terminal; and can be integrated to multiple systems to provide services for other organizations. However, in the present disclosure, it is easy to reduce precision of subsequent detection due to a device location abnormality, causing poor detection accuracy and degrading user experience. In addition, an existing life early warning system based on sensor acquisition cannot update a detection parameter of a sign detection module by itself, and maintenance personnel need to frequently update the system. In this case, the present disclosure proposes a life early warning system based on sensor acquisition.

### SUMMARY

The present disclosure aims to resolve disadvantages in the conventional technology and propose a life early warning system based on sensor acquisition.

To achieve the above objective, the present disclosure adopts the following technical solutions:

A life early warning system based on sensor acquisition includes a sign sensor, a camera, a location analysis module, an attachment location correction module, a sign detection module, an alarm feedback module, a detection optimizer, a medical comparison library, a medical platform, and a client, where
the sign sensor is configured to attach to a specified location of a user body, and acquire user sign information in real time;
the camera is configured to acquire an attachment location of the sign sensor, and generate image information;
the location analysis module is configured to perform cascading analysis on acquired image information, and generate location information of each sign sensor;
the attachment location correction module is configured to receive a location of each sign sensor, perform a determining and judgment action, prompt a user of information about a sign sensor with an attachment location error, and remind the user to make a correction;
the sign detection module is configured to receive user sign information, and fetch related data from the medical comparison library to perform comparison recording to generate a sign report;
the alarm feedback module is configured to send early warning information to the user according to the sign report;
the detection optimizer is configured to periodically perform optimization adjustment on the sign detection module;
the medical comparison library is configured to store sign index information;
the medical platform is configured to receive the sign report of the user, feed the sign report back to a corresponding physician for verification, and feed back a conditioning scheme; and
the client is configured for the user to log in, upload basic personal data, and view a personal sign report and the conditioning scheme.

In a further solution of the present disclosure, specific steps of cascading analysis of the location analysis module are as follows:
step (1): the location analysis module performs frame-by-frame extraction on the image information to obtain detection pictures, receives groups of detection pictures by using a first-level target detection network, constructs a picture data set according to different sizes of the groups of detection pictures, scales the groups of detection pictures according to resolutions specified by a system or manually set, and infers the groups of detection pictures with different resolutions;
step (2): the location analysis module aggregates inference results to perform non-maximum suppression to obtain feature data, sends the extracted feature data to a bidirectional feature pyramid to perform feature fusion, and performs classification and regression on output of a bidirectional feature pyramid network (BiFPN) to output a detection frame, a category, and a score;
step (3): the location analysis module obtains a resolution of an input detection picture and a width and a depth of a target detection network as to-be-optimized parameters, and performs a large quantity of searches on an architecture of the target detection network, to search, when a quantity of parameters of the target detection network is less than a value, for a parameter that enables detection to have a highest precision rate; and
step (4): the location analysis module acquires information about sensor detection frames in the detection pictures, generates coordinates corresponding to the detection frames, performs zoom-in clipping on related detection pictures, acquires groups of sensor pictures generated after zoom-in clipping, stores the groups of sensor pictures, uses a second-level target detection network to filter, by using a reverse polish notation (RPN), out a simple negative sample in the group of sensor pictures that belongs to a background, selects areas possibly including targets to perform classification and regression, and obtains specific locations of sensors in the groups of sensor pictures through zoom-in clipping.

In a further solution of the present disclosure, calculation formulas of the zoom-in clipping in step (4) are as follows:

x1′=max(x1−|x2−x1|*e,0) (1)

x2′=min(x2+|x2−x1|*e,width) (2)

y1′=max(y1−|y2−y1|*e,0) (3)

y2′=min(y2+|y2−y1|*e,height) (4), where

width and height respectively represent a width and a height of a to-be-detected picture, in pixel, e represents a zoom-in ratio, and e progressively increases sequentially from 0 to 0.8 by 0.2, and x1, x2, y1, and y2 are coordinates of related detection frames.

In a further solution of the present disclosure, specific steps for determining the location of each sign sensor by the attachment location correction module are as follows:
step 1: the attachment location correction module receives the basic personal data sent by the client, constructs a related human body simulation model according to the basic personal data, and matches the location information of each sign sensor obtained through the cascading analysis to the human body simulation model; and
step 2: the attachment location correction module determines a location of each current sign sensor according to a location specified by a default sign sensor of the system, marks a sign sensor having a deviation, forbids the user from performing subsequent sign detection, feeds back an abnormal sign sensor to the user by using an external display, and reminds the user to make a correction.

In a further solution of the present disclosure, specific steps of performing comparison recording by the sign detection module are as follows:
step 1: the sign detection module receives the user sign information, classifies the user sign information according to pulse rate, blood pressure, respiration, pupil, and corneal reflex, and generates a sign record table to record each group of acquired user sign information; and
step 2: the sign detection module extracts sign index information from the medical comparison library, records the sign index information into the sign record table, compares each piece of acquired user sign information with the sign index information, and records a body status of the user to generate a corresponding user sign report.

In a further solution of the present disclosure, specific steps of performing optimization adjustment by the detection optimizer are as follows:
S1.1: the detection optimizer receives a physician verification result fed back by the medical platform, gathers and integrates multiple groups of verification results to generate an observation dataset, and selects one piece of observation data from the observation dataset as verification data;
S1.2: the detection optimizer uses remaining observation data to fit a test model, verifies precision of the test model by using the selected verification data, and calculates a prediction capability of the test model by using a root-mean-square error (RMSE), in this way, n times are repeated to perform parameter optimization on a generated precision parameter;
S1.3: the detection optimizer initializes a parameter range, sets a learning rate η=[0.0001, 0.1], lists all possible data results, selects any subset as a test set for each group of data, uses a remaining subset as a training set, predicts the test set after the test model is trained, counts an RMSE of a test result, and selects a combination parameter corresponding to a smallest RMSE as an optimal parameter in a data interval until all data is predicted once; and
S1.4: the detection optimizer receives a detection parameter of the sign detection module in real time, filters out a detection parameter with a poor detection capability, classifies detection efficiency of the sign detection module into a training set and a test set, performs normalization processing on the training set by using the optimal parameter to obtain a training sample, trains the sign detection module by using a long-term iteration method, and imports the test set into a trained sign detection module to obtain a detection curve of the sign detection module.

In a further solution of the present disclosure, a specific calculation formula of the RMSE in S1.2 is as follows:

$$RMSE = \sqrt{\frac{{\sum }_{i = 1}^{n} {( E ( {y}_{i} ) - {y}_{i} )}^{2}}{n}} , \qquad ( 5 )$$

where

E(yᵢ) indicates an i^{th} actual observation value, yᵢ is an i^{th} predicted value inversed by the test model, and n is a total amount of observation data.

Compared with the conventional technology, the present disclosure has the following beneficial effects:
1. In comparison with a previous life early warning system, the system of the present disclosure has following advantages. The camera is configured to acquire the attachment location of the sign sensor. The location analysis module performs frame-by-frame extraction on the image information acquired by the camera to obtain the detection pictures; scales the groups of detection pictures according to the resolutions specified by the system or manually set by using the first-level target detection network; obtains the feature data of the detection pictures, and obtains the detection frames through classification and regression; and performs the zoom-in clipping on the related detection pictures, acquires the groups of sensor pictures generated after the zoom-in clipping, stores the groups of sensor pictures, uses the second-level target detection network to filter, by using the RPN, out the simple negative sample in the group of sensor pictures that belongs to the background, selects the areas possibly including the targets to perform classification and regression, and obtains the specific locations of the sensors in the groups of sensor pictures through the zoom-in clipping. The attachment location correction module determines whether the attachment location of each sign sensor complies with a usage specification. A location of the sign sensor is analyzed by using a cascade network, which can ensure accuracy of the attachment location of each sensor, avoid reduction in precision of subsequent detection due to a sensor location abnormality, greatly improve detection accuracy, further prevent an abnormal sign report due to misusing by a child, and improve user experience.
2. In the present disclosure, the detection optimizer receives the physician verification result fed back by the medical platform, gathers and integrates the multiple groups of verification results to generate the observation dataset, and selects one piece of observation data from the observation dataset as the verification data; uses the remaining observation data to fit the test model, verifies the precision of the test model by using the selected verification data, and calculates the prediction capability of the test model by using the RMSE, in this way, the n times are repeated to perform parameter optimization on the generated precision parameter; and selects the combination parameter corresponding to the smallest RMSE as the optimal parameter in the data interval; and the detection optimizer receives the detection parameter of the sign detection module in real time, filters out the detection parameter with a poor detection capability, classifies the detection efficiency of the sign detection module into the training set and the test set, performs normalization processing on the training set by using the optimal parameter to obtain the training sample, trains the sign detection module by using the long-term iteration method, and imports the test set into the trained sign detection module to obtain the detection curve of the sign detection module. A detection parameter of the sign detection module can be continuously updated, continuously improving accuracy of a sign report, effectively reducing a system update frequency of maintenance personnel, and reducing workload of the maintenance personnel.

### BRIEF DESCRIPTION OF THE DRAWINGS

The drawings are provided for further understanding of the present disclosure and constitute a part of the specification. The drawings, together with the embodiments of the present disclosure, are intended to explain the present disclosure, rather than to limit the present disclosure.

FIG. 1 is a system block diagram of a life early warning system based on sensor acquisition according to the present disclosure; and

FIG. 2 is a block diagram of a determining procedure of a location analysis module of a life early warning system based on sensor acquisition according to the present disclosure.

### DETAILED DESCRIPTION OF THE EMBODIMENTS

The technical solutions of the embodiments of the present disclosure are clearly and completely described below with reference to the drawings in the embodiments of the present disclosure. Apparently, the described embodiments are merely a part rather than all of the embodiments of the present disclosure.

### Embodiment 1

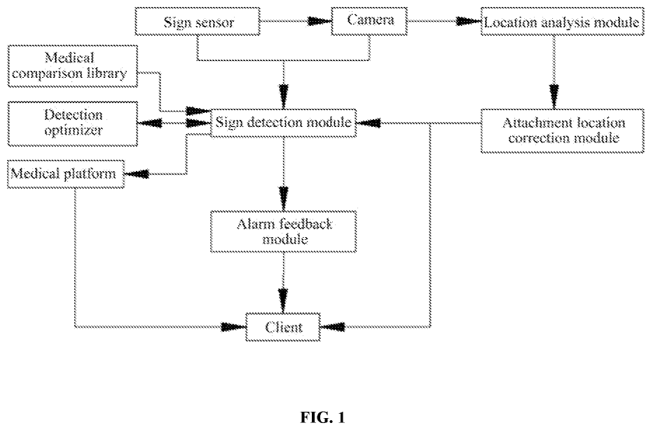

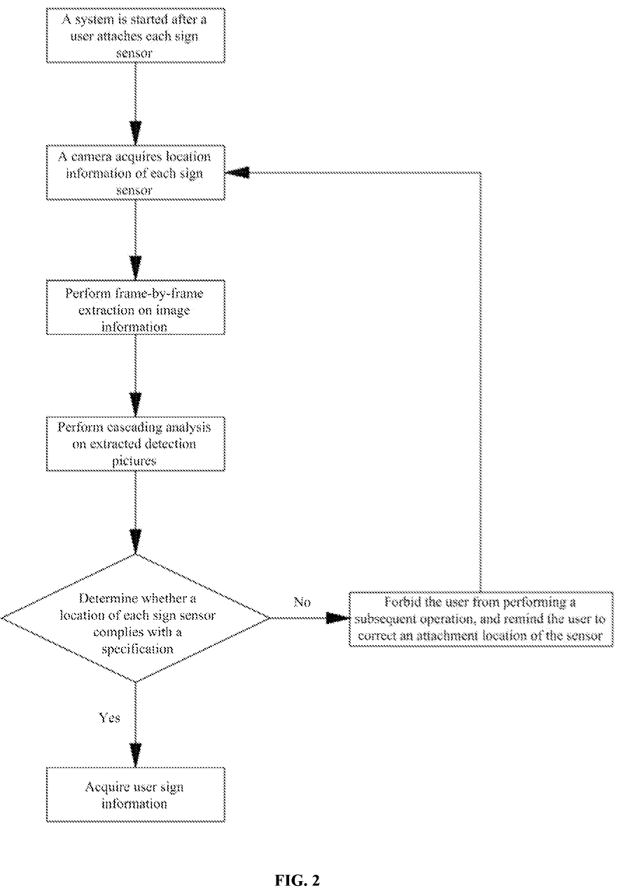

Referring to FIG. 1 and FIG. 2, a life early warning system based on sensor acquisition includes a sign sensor, a camera, a location analysis module, an attachment location correction module, a sign detection module, an alarm feedback module, a detection optimizer, a medical comparison library, a medical platform, and a client.

The sign sensor is configured to attach to a specified location of a user body, and acquire user sign information in real time. The camera is configured to acquire an attachment location of the sign sensor, and generate image information.

The location analysis module is configured to perform cascading analysis on acquired image information, and generate location information of each sign sensor.

Specifically, referring to FIG. 2, it can be learned that the location analysis module performs frame-by-frame extraction on the image information to obtain detection pictures, receives groups of detection pictures by using a first-level target detection network, constructs a picture data set according to different sizes of the groups of detection pictures, scales the groups of detection pictures according to resolutions specified by a system or manually set, and infers the groups of detection pictures with different resolutions; aggregates inference results to perform non-maximum suppression to obtain feature data, sends the extracted feature data to a bidirectional feature pyramid to perform feature fusion, and performs classification and regression on output of a BiFPN to output a detection frame, a category, and a score; obtains a resolution of an input detection picture and a width and a depth of a target detection network as to-be-optimized parameters, and performs a large quantity of searches on an architecture of the target detection network, to search, when a quantity of parameters of the target detection network is less than a value, for a parameter that enables detection to have a highest precision rate; and acquires information about sensor detection frames in the detection pictures, generates coordinates corresponding to the detection frames, performs zoom-in clipping on related detection pictures, acquires groups of sensor pictures generated after zoom-in clipping, stores the groups of sensor pictures, uses a second-level target detection network to filter, by using an RPN, out a simple negative sample in the group of sensor pictures that belongs to a background, selects areas possibly including targets to perform classification and regression, and obtains specific locations of sensors in the groups of sensor pictures through zoom-in clipping. A location of the sign sensor is analyzed by using a cascade network, which can ensure accuracy of the attachment location of each sensor, avoid reduction in precision of subsequent detection due to a sensor location abnormality, greatly improve detection accuracy, further prevent an abnormal sign report due to misusing by a child, and improve user experience.

It should be further noted that calculation formulas of the zoom-in clipping are as follows:

x1′=max(x1−|x2−x1|*e,0) (1)

x2′=min(x2+|x2−x1|*e,width) (2)

y1′=max(y1−|y2−y1|*e,0) (3)

y2′=min(y2+|y2−y1|*e,height) (4), where

width and height respectively represent a width and a height of a to-be-detected picture, in pixel, e represents a zoom-in ratio, and e progressively increases sequentially from 0 to 0.8 by 0.2, and x1, x2, y1, and y2 are coordinates of related detection frames.

The attachment location correction module is configured to receive a location of each sign sensor, perform a determining and judgment action, prompt a user of information about a sign sensor with an attachment location error, and remind the user to make a correction.

Specifically, the attachment location correction module receives basic personal data sent by the client, constructs a related human body simulation model according to the basic personal data, and matches the location information of each sign sensor obtained through the cascading analysis to the human body simulation model; and the attachment location correction module determines a location of each current sign sensor according to a location specified by a default sign sensor of the system, marks a sign sensor having a deviation, forbids the user from performing subsequent sign detection, feeds back an abnormal sign sensor to the user by using an external display, and reminds the user to make a correction.

The sign detection module is configured to receive user sign information, and fetch related data from the medical comparison library to perform comparison recording to generate a sign report. The alarm feedback module is configured to send early warning information to the user according to the sign report.

Specifically, the sign detection module receives the user sign information, classifies the user sign information according to pulse rate, blood pressure, respiration, pupil, and corneal reflex, and generates a sign record table to record each group of acquired user sign information; and extracts sign index information from the medical comparison library, records the sign index information into the sign record table, compares each piece of acquired user sign information with the sign index information, and records a body status of the user to generate a corresponding user sign report.

### Embodiment 2

Referring to FIG. 1, a life early warning system based on sensor acquisition includes a sign sensor, a camera, a location analysis module, an attachment location correction module, a sign detection module, an alarm feedback module, a detection optimizer, a medical comparison library, a medical platform, and a client.

The detection optimizer is configured to periodically perform optimization adjustment on the sign detection module.

Specifically, the detection optimizer receives a physician verification result fed back by the medical platform, gathers and integrates multiple groups of verification results to generate an observation dataset, and selects one piece of observation data from the observation dataset as verification data; uses remaining observation data to fit a test model, verifies precision of the test model by using the selected verification data, and calculates a prediction capability of the test model by using an RMSE, in this way, n times are repeated to perform parameter optimization on a generated precision parameter; and initializes a parameter range, sets a learning rate η=[0.0001, 0.1], lists all possible data results, selects any subset as a test set for each group of data, uses a remaining subset as a training set, predicts the test set after the test model is trained, counts an RMSE of a test result, and selects a combination parameter corresponding to a smallest RMSE as an optimal parameter in a data interval until all data is predicted once; and the detection optimizer receives a detection parameter of the sign detection module in real time, filters out a detection parameter with a poor detection capability, classifies detection efficiency of the sign detection module into a training set and a test set, performs normalization processing on the training set by using the optimal parameter to obtain a training sample, trains the sign detection module by using a long-term iteration method, and imports the test set into a trained sign detection module to obtain a detection curve of the sign detection module.

It should be further noted that a specific calculation formula of the RMSE is as follows:

$$RMSE = \sqrt{\frac{{\sum }_{i = 1}^{n} {( E ( {y}_{i} ) - {y}_{i} )}^{2}}{n}} , \qquad ( 5 )$$

where

E(yᵢ) indicates an i^{th} actual observation value, yᵢ is an i^{th} predicted value inversed by the test model, and n is a total amount of observation data.

The medical comparison library is configured to store sign index information.

It should be further noted that the sign index information specifically includes: A body temperature index is 36-37 degrees, a pulse rate index for adults is 60-100 beats per minute, a pulse rate index for the elderly is 55-60 beats per minute, a pulse rate index for infants and young children is 90-140 beats per minute, a pulse rate index for children is 80-90 beats per minute, a respiratory rate index is 18-22 times per minute, a blood pressure index includes that a systolic blood pressure is 90-140 millimeter of mercury and a diastolic blood pressure is 60-90 millimeter of mercury, and a pupil index is 2-5 mm in diameter.

The medical platform is configured to receive the sign report of the user, feed the sign report back to a corresponding physician for verification, and feed back a conditioning scheme.

The client is configured for the user to log in, upload the basic personal data, and view a personal sign report and the conditioning scheme.

It should be further noted that the user may select application programs by using the client, and the client stores each application program in a form of a least recently used (LRU) linked list according to selection information of the user. A specific storage principle of the LRU linked list is as follows. First, headers of each group of start linked lists are further linked by using the LRU linked list according to an LRU sequence of functional programs; information about a least used application program is acquired; and a start linked list of the application program is arranged in a first place of the LRU linked list, and sorting is performed in sequence. Before access information is traced in a startup phase of the application program, the client clears access bits of all update page entries before the application program is started. If an application program is accessed during startup of the application program, the client adds this page to the start linked list. Before startup time of the application program ends, the client re-checks all access bits of the application program. If the application program is accessed at another stage, the application program is deleted from the start linked list and moved to a regular LRU linked list, and sorting and updating are performed on each group of application programs in the start linked list after determining is completed.

What is claimed is:

1. A life early warning system based on sensor acquisition, comprising a sign sensor, a camera, a location analysis module, an attachment location correction module, a sign detection module, an alarm feedback module, a detection optimizer, a medical comparison library, a medical platform, and a client, wherein
the sign sensor is configured to attach to a specified location of a user body, and acquire user sign information in real time;
the camera is configured to acquire an attachment location of the sign sensor, and generate image information;
the location analysis module is configured to perform cascading analysis on acquired image information, and generate location information of each sign sensor;
the attachment location correction module is configured to receive a location of each sign sensor, perform a determining and judgment action, prompt a user of information about a sign sensor with an attachment location error, and remind the user to make a correction;
the sign detection module is configured to receive user sign information, and fetch related data from the medical comparison library to perform comparison recording to generate a sign report;
the alarm feedback module is configured to send early warning information to the user according to the sign report;
the detection optimizer is configured to periodically perform optimization adjustment on the sign detection module;
the medical comparison library is configured to store sign index information;
the medical platform is configured to receive the sign report of the user, feed the sign report back to a corresponding physician for verification, and feed back a conditioning scheme; and
the client is configured for the user to log in, upload basic personal data, and view a personal sign report and the conditioning scheme;
wherein the client is configured to store each application program in a form of a least recently used (LRU) linked list according to selection information of the user, wherein a specific storage principle of the LRU linked list is as follows: first, headers of each group of start linked lists are further linked by using the LRU linked list according to an LRU sequence of functional programs; information about a least used application program is acquired; and a start linked list of the application program is arranged in a first place of the LRU linked list, and sorting is performed in sequence; before access information is traced in a startup phase of the application program, the client clears access bits of all update page entries before the application program is started; if an application program is accessed during startup of the application program, the client adds a page to the start linked list; before startup time of the application program ends, the client re-checks all access bits of the application program; if the application program is accessed at another stage, the application program is deleted from the start linked list and moved to a regular LRU linked list, and sorting and updating are performed on each group of application programs in the start linked list after determining is completed;
specific steps of cascading analysis of the location analysis module are as follows:
step (1): the location analysis module performs frame-by-frame extraction on the image information to obtain detection pictures, receives groups of detection pictures by using a first-level target detection network, constructs a picture data set according to different sizes of the groups of detection pictures, scales the groups of detection pictures according to resolutions specified by a system or manually set, and infers the groups of detection pictures with different resolutions;
step (2): the location analysis module aggregates inference results to perform non-maximum suppression to obtain feature data, sends the extracted feature data to a bidirectional feature pyramid to perform feature fusion, and performs classification and regression on output of a bidirectional feature pyramid network (BiFPN) to output a detection frame, a category, and a score;
step (3): the location analysis module obtains a resolution of an input detection picture and a width and a depth of a target detection network as to-be-optimized parameters, and performs a large quantity of searches on an architecture of the target detection network, to search, when a quantity of parameters of the target detection network is less than a value, for a parameter that enables detection to have a highest precision rate; and
step (4): the location analysis module acquires information about sensor detection frames in the detection pictures, generates coordinates corresponding to the detection frames, performs zoom-in clipping on related detection pictures, acquires groups of sensor pictures generated after zoom-in clipping, stores the groups of sensor pictures, uses a second-level target detection network to filter, by using a reverse polish notation (RPN), out a simple negative sample in the group of sensor pictures that belongs to a background, selects areas possibly comprising targets to perform classification and regression, and obtains specific locations of sensors in the groups of sensor pictures through zoom-in clipping;
wherein specific steps for determining the location of each sign sensor by the attachment location correction module are as follows:
step 1: the attachment location correction module receives the basic personal data sent by the client, constructs a related human body simulation model according to the basic personal data, and matches the location information of each sign sensor obtained through the cascading analysis to the human body simulation model; and
step 2: the attachment location correction module determines a location of each current sign sensor according to a location specified by a default sign sensor of the system, marks a sign sensor having a deviation, forbids the user from performing subsequent sign detection, feeds back an abnormal sign sensor to the user by using an external display, and reminds the user to make a correction.

2. The life early warning system based on sensor acquisition according to claim 1, wherein calculation formulas of the zoom-in clipping in step (4) are as follows:
x1′=max(x1−|x2−x1|*e,0) (1)
x2′=min(x2+|x2−x1|*e,width) (2)
y1′=max(y1−|y2−y1|*e,0) (3)
y2′=min(y2+|y2−y1|*e,height) (4), where
width and height respectively represent a width and a height of a to-be-detected picture, in pixel, e represents a zoom-in ratio, and e progressively increases sequentially from 0 to 0.8 by 0.2, and x1, x2, y1, and y2 are coordinates of related detection frames.

3. The life early warning system based on sensor acquisition according to claim 1, wherein specific steps of performing comparison recording by the sign detection module are as follows:
step 1: the sign detection module receives the user sign information, classifies the user sign information according to pulse rate, blood pressure, respiration, pupil, and corneal reflex, and generates a sign record table to record each group of acquired user sign information; and
step 2: the sign detection module extracts sign index information from the medical comparison library, records the sign index information into the sign record table, compares each piece of acquired user sign information with the sign index information, and records a body status of the user to generate a corresponding user sign report.

4. The life early warning system based on sensor acquisition according to claim 1, wherein specific steps of performing optimization adjustment by the detection optimizer are as follows:
S1.1: the detection optimizer receives a physician verification result fed back by the medical platform, gathers and integrates multiple groups of verification results to generate an observation dataset, and selects one piece of observation data from the observation dataset as verification data;
S1.2: the detection optimizer uses remaining observation data to fit a test model, verifies precision of the test model by using the selected verification data, and calculates a prediction capability of the test model by using a root-mean-square error (RMSE), in this way, n times are repeated to perform parameter optimization on a generated precision parameter;
S1.3: the detection optimizer initializes a parameter range, sets a learning rate η=[0.0001, 0.1], lists all possible data results, selects any subset as a test set for each group of data, uses a remaining subset as a training set, predicts the test set after the test model is trained, counts an RMSE of a test result, and selects a combination parameter corresponding to a smallest RMSE as an optimal parameter in a data interval until all data is predicted once; and
S1.4: the detection optimizer receives a detection parameter of the sign detection module in real time, filters out a detection parameter with a poor detection capability, classifies detection efficiency of the sign detection module into a training set and a test set, performs normalization processing on the training set by using the optimal parameter to obtain a training sample, trains the sign detection module by using a long-term iteration method, and imports the test set into a trained sign detection module to obtain a detection curve of the sign detection module.

5. The life early warning system based on sensor acquisition according to claim 4, wherein a specific calculation formula of the RMSE in S1.2 is as follows:
$$RMSE = \sqrt{\frac{{\sum }_{i = 1}^{n} {( E ( {y}_{i} ) - {y}_{i} )}^{2}}{n}} , \qquad ( 5 )$$
wherein
E(yᵢ) indicates an i^{th} actual observation value, yᵢ is an i^{th} predicted value inversed by the test model, and n is a total amount of observation data.
